(19) United States
(12) Patent Application Publication
(10) Pub. No.: US 2021/0128007 A1
(43) Pub. Date: May 6, 2021

(54) METHOD AND APPARATUS FOR FILTERING ELECTROCARDIOGRAM SIGNAL

(71) Applicant: TENCENT AMERICA LLC, Palo Alto, CA (US)

(72) Inventors: Xiaozhong Chen, Cedarburg, WI (US); Kun Wang, San Jose, CA (US); Wei Fan, New York, NY (US)

(73) Assignee: TENCENT AMERICA LLC, Palo Alto, CA (US)

(21) Appl. No.: 16/670,214

(22) Filed: Oct. 31, 2019

(51) Int. Cl. A61B 5/0472; A61B 5/00

(52) U.S. Cl. CPC A61B 5/0472; A61B 5/725; A61B 5/7253

(57) ABSTRACT

A method of filtering an electrocardiogram (ECG) signal includes obtaining the ECG signal; applying a first transformation to the ECG signal to generate a transformed ECG signal; filtering the transformed ECG signal to generate a filtered transformed ECG signal; and applying a second transformation to the filtered transformed ECG signal to generate a filtered ECG signal.

### BACKGROUND

Electrocardiogram (ECG) is a popular technique used to monitor the status of heart. In an ECG test, electromagnetic activity occurring in cardiac muscle is recorded, for example from a from body surface, and the recorded signal is interpreted by ECG experts or an automatic analysis system to estimate the status of the heart.

FIG. 1 illustrates a schematic of an example of an ECG signal 100. Referring to FIG. 1, an ECG signal may include, for example P-wave 110, PR interval 120, PR segment 130, QRS complex 140, QT interval 150, ST segment 160, and T wave 170. Each of these elements may indicate important information about different stages of the propagation of a cardiac excitation in the heart. For example, any deviation of these ECG segments away from their normal status may indicate potential disease or problem in the heart. As an example, an elevation or depression of the ST segment 160 may indicate myocardium infarction, and a morphology change of the QRS complex 140 may indicate abnormalities in the intraventricular conduction system.

It is important to maintain the morphology of ECG during the signal collecting and processing process so that any diagnosis derived from the ECG is correct. However, an ECG signal recorded from body surface is very likely to be contaminated by electromagnetic signals generated by sources other than the heart, such as skeletal muscles. This type of noise is usually known as muscle noise, and makes automatic ECG analysis software susceptible to errors. Removing muscle noise from ECG recordings without causing significant alternation to the real ECG signal is an important component of an automatic ECG analysis system.

### SUMMARY

According to an embodiment, a method of filtering an electrocardiogram (ECG) signal includes obtaining the ECG signal; applying a first transformation to the ECG signal to generate a transformed ECG signal; filtering the transformed ECG signal to generate a filtered transformed ECG signal; and applying a second transformation to the filtered transformed ECG signal to generate a filtered ECG signal.

According to an embodiment, a device for filtering an electrocardiogram (ECG) signal includes: at least one memory configured to store program code; and at least one processor configured to read the program code and operate as instructed by the program code, the program code including: obtaining code configured to cause the at least one processor to obtain the ECG signal; first transformation code configured to cause the at least one processor to apply a first transformation to the ECG signal to generate a transformed ECG signal; filtering code configured to cause the at least one processor to filter the transformed ECG signal to generate a filtered transformed ECG signal; second transformation code configured to cause the at least one processor to apply a second transformation to the filtered transformed ECG signal to generate a filtered ECG signal.

According to an embodiment, a non-transitory computer-readable medium stores instructions including one or more instructions that, when executed by one or more processors of a device for filtering an electrocardiogram (ECG) signal, cause the one or more processors to: obtain the ECG signal; apply a first transformation to the ECG signal to generate a transformed ECG signal; filter the transformed ECG signal to generate a filtered transformed ECG signal; and apply a second transformation to the filtered transformed ECG signal to generate a filtered ECG signal.

### BRIEF DESCRIPTION OF THE DRAWINGS

FIG. 1 is a schematic representation of an example of an ECG signal;

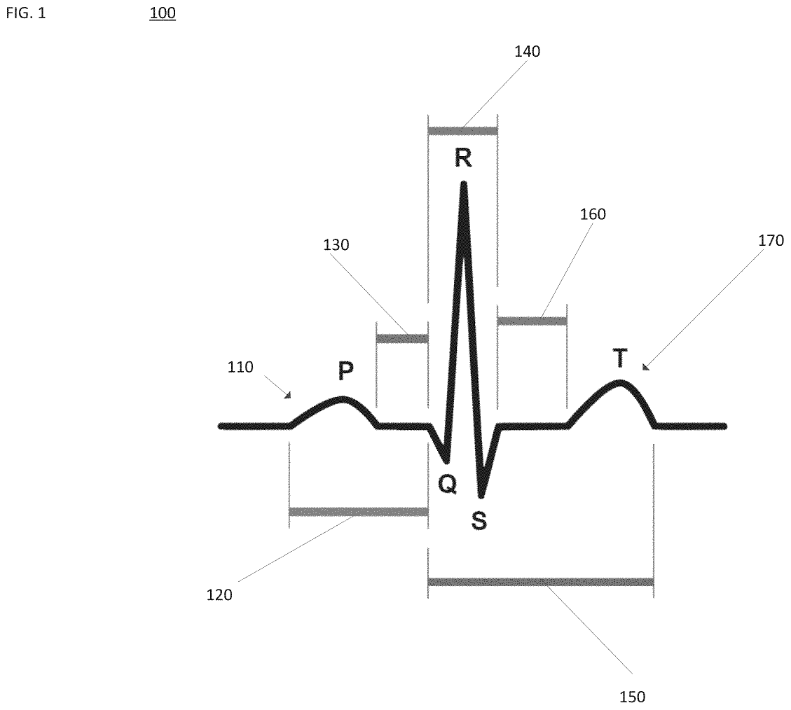

FIG. 2 is a diagram of an example environment in which systems and/or methods, described herein, may be implemented;

FIG. 3 is a diagram of example components of one or more devices of FIG. 2;

FIGS. 4A-4C illustrate an example of a Fast Fourier Transform technique of noise reduction;

FIG. 5 illustrates a signal filtering schema according to an embodiment;

FIG. 6 illustrates a result of a first transformation according to an embodiment;

FIG. 7 illustrates a result of a second transformation according to an embodiment; and

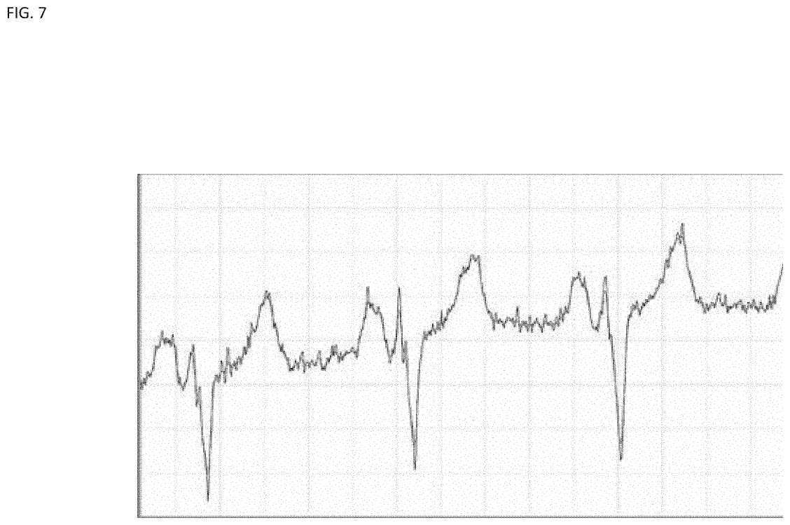

FIG. 8 is a flow chart of an example process for filtering an ECG signal.

### DETAILED DESCRIPTION

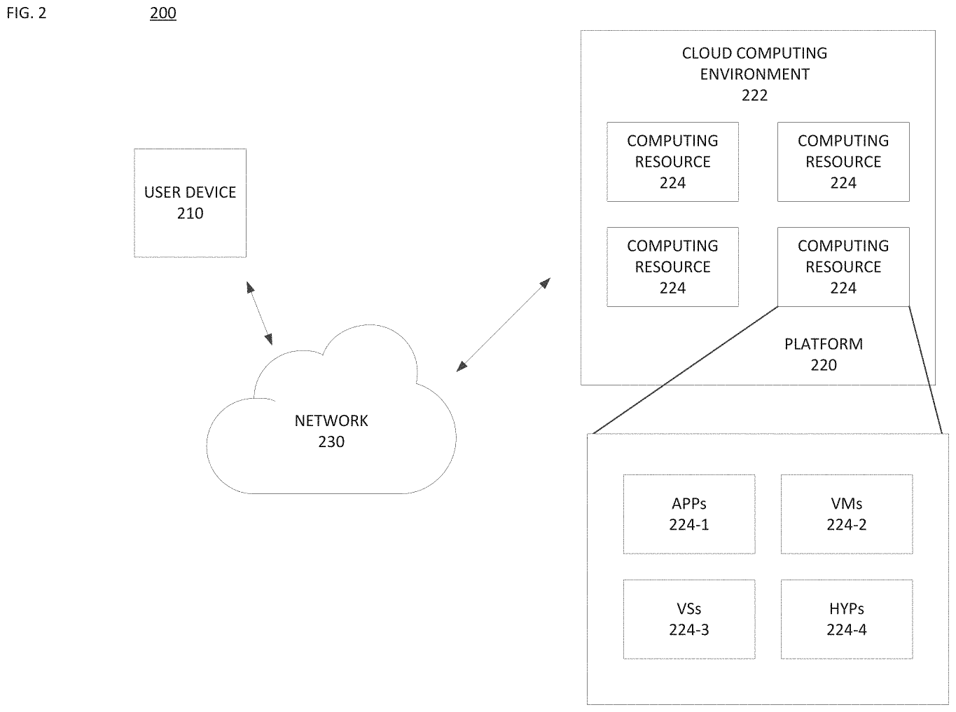

FIG. 2 is a diagram of an example environment 200 in which systems and/or methods, described herein, may be implemented. As shown in FIG. 2, environment 200 may include a user device 210, a platform 220, and a network 230. Devices of environment 200 may interconnect via wired connections, wireless connections, or a combination of wired and wireless connections.

User device 210 includes one or more devices capable of receiving, generating, storing, processing, and/or providing information associated with platform 220. For example, user device 210 may include a computing device (e.g., a desktop computer, a laptop computer, a tablet computer, a handheld computer, a smart speaker, a server, etc.), a mobile phone (e.g., a smart phone, a radiotelephone, etc.), a wearable device (e.g., a pair of smart glasses or a smart watch), or a similar device. In some implementations, user device 210 may receive information from and/or transmit information to platform 220. In some embodiments, user device 210 may include or be operable to communicate with a sensor such as an electromagnetic sensor which may be used to measure electromagnetic signals, for example at a surface of a body.

Platform 220 includes one or more devices capable of obtaining and filtering an ECG signal, as described elsewhere herein. In some implementations, platform 220 may include a cloud server or a group of cloud servers. In some implementations, platform 220 may be designed to be modular such that certain software components may be swapped in or out depending on a particular need. As such, platform 220 may be easily and/or quickly reconfigured for different uses.

In some implementations, as shown, platform 220 may be hosted in cloud computing environment 222. Notably, while implementations described herein describe platform 220 as being hosted in cloud computing environment 222, in some implementations, platform 220 is not be cloud-based (i.e., may be implemented outside of a cloud computing environment) or may be partially cloud-based.

Cloud computing environment 222 includes an environment that hosts platform 220. Cloud computing environment 222 may provide computation, software, data access, storage, etc. services that do not require end-user (e.g., user device 210) knowledge of a physical location and configuration of system(s) and/or device(s) that hosts platform 220. As shown, cloud computing environment 222 may include a group of computing resources 224 (referred to collectively as “computing resources 224” and individually as “computing resource 224”).

Computing resource 224 includes one or more personal computers, workstation computers, server devices, or other types of computation and/or communication devices. In some implementations, computing resource 224 may host platform 220. The cloud resources may include compute instances executing in computing resource 224, storage devices provided in computing resource 224, data transfer devices provided by computing resource 224, etc. In some implementations, computing resource 224 may communicate with other computing resources 224 via wired connections, wireless connections, or a combination of wired and wireless connections.

As further shown in FIG. 2, computing resource 224 includes a group of cloud resources, such as one or more applications (“APPs”) 224-1, one or more virtual machines (“VMs”) 224-2, virtualized storage (“VSs”) 224-3, one or more hypervisors (“HYPs”) 224-4, or the like.

Application 224-1 includes one or more software applications that may be provided to or accessed by user device 210 and/or sensor device 220. Application 224-1 may eliminate a need to install and execute the software applications on user device 210. For example, application 224-1 may include software associated with platform 220 and/or any other software capable of being provided via cloud computing environment 222. In some implementations, one application 224-1 may send/receive information to/from one or more other applications 224-1, via virtual machine 224-2.

Virtual machine 224-2 includes a software implementation of a machine (e.g., a computer) that executes programs like a physical machine. Virtual machine 224-2 may be either a system virtual machine or a process virtual machine, depending upon use and degree of correspondence to any real machine by virtual machine 224-2. A system virtual machine may provide a complete system platform that supports execution of a complete operating system (“OS”). A process virtual machine may execute a single program, and may support a single process. In some implementations, virtual machine 224-2 may execute on behalf of a user (e.g., user device 210), and may manage infrastructure of cloud computing environment 222, such as data management, synchronization, or long-duration data transfers.

Virtualized storage 224-3 includes one or more storage systems and/or one or more devices that use virtualization techniques within the storage systems or devices of computing resource 224. In some implementations, within the context of a storage system, types of virtualizations may include block virtualization and file virtualization. Block virtualization may refer to abstraction (or separation) of logical storage from physical storage so that the storage system may be accessed without regard to physical storage or heterogeneous structure. The separation may permit administrators of the storage system flexibility in how the administrators manage storage for end users. File virtualization may eliminate dependencies between data accessed at a file level and a location where files are physically stored. This may enable optimization of storage use, server consolidation, and/or performance of non-disruptive file migrations.

Hypervisor 224-4 may provide hardware virtualization techniques that allow multiple operating systems (e.g., “guest operating systems”) to execute concurrently on a host computer, such as computing resource 224. Hypervisor 224-4 may present a virtual operating platform to the guest operating systems, and may manage the execution of the guest operating systems. Multiple instances of a variety of operating systems may share virtualized hardware resources.

Network 230 includes one or more wired and/or wireless networks. For example, network 230 may include a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of devices and networks shown in FIG. 2 are provided as an example. In practice, there may be additional devices and/or networks, fewer devices and/or networks, different devices and/or networks, or differently arranged devices and/or networks than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of environment 200 may perform one or more functions described as being performed by another set of devices of environment 200.

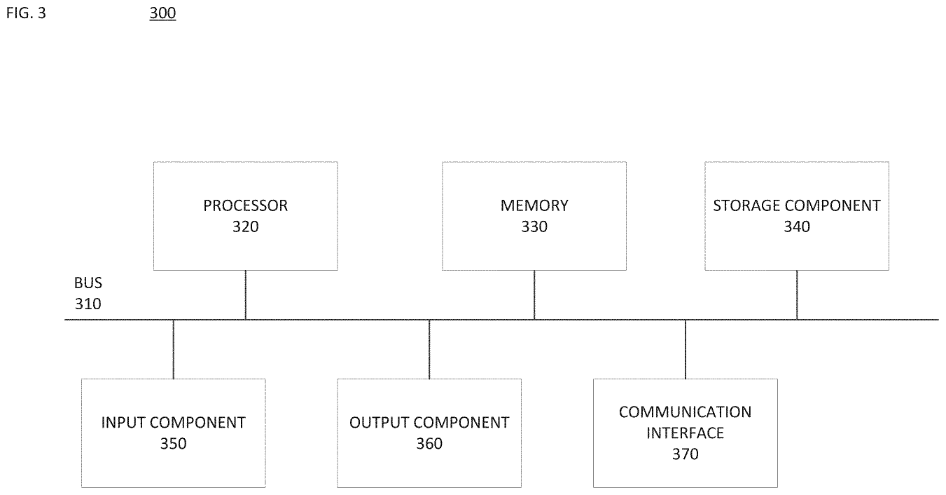

FIG. 3 is a diagram of example components of a device 300. Device 300 may correspond to user device 210 and/or platform 220. As shown in FIG. 3, device 300 may include a bus 310, a processor 320, a memory 330, a storage component 340, an input component 350, an output component 360, and a communication interface 370.

Bus 310 includes a component that permits communication among the components of device 300. Processor 320 is implemented in hardware, firmware, or a combination of hardware and software. Processor 320 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 320 includes one or more processors capable of being programmed to perform a function. Memory 330 includes a random access memory (RAM), a read only memory (ROM), and/or another type of dynamic or static storage device (e.g., a flash memory, a magnetic memory, and/or an optical memory) that stores information and/or instructions for use by processor 320.

Storage component 340 stores information and/or software related to the operation and use of device 300. For example, storage component 340 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

Input component 350 includes a component that permits device 300 to receive information, such as via user input (e.g., a touch screen display, a keyboard, a keypad, a mouse, a button, a switch, and/or a microphone). Additionally, or alternatively, input component 350 may include a sensor for sensing information (e.g., a global positioning system (GPS) component, an accelerometer, a gyroscope, and/or an actuator). Output component 360 includes a component that provides output information from device 300 (e.g., a display, a speaker, and/or one or more light-emitting diodes (LEDs)).

Communication interface 370 includes a transceiver-like component (e.g., a transceiver and/or a separate receiver and transmitter) that enables device 300 to communicate with other devices, such as via a wired connection, a wireless connection, or a combination of wired and wireless connections. Communication interface 370 may permit device 300 to receive information from another device and/or provide information to another device. For example, communication interface 370 may include an Ethernet interface, an optical interface, a coaxial interface, an infrared interface, a radio frequency (RF) interface, a universal serial bus (USB) interface, a Wi-Fi interface, a cellular network interface, or the like.

Device 300 may perform one or more processes described herein. Device 300 may perform these processes in response to processor 320 executing software instructions stored by a non-transitory computer-readable medium, such as memory 330 and/or storage component 340. A computer-readable medium is defined herein as a non-transitory memory device. A memory device includes memory space within a single physical storage device or memory space spread across multiple physical storage devices.

Software instructions may be read into memory 330 and/or storage component 340 from another computer-readable medium or from another device via communication interface 370. When executed, software instructions stored in memory 330 and/or storage component 340 may cause processor 320 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 3 are provided as an example. In practice, device 300 may include additional components, fewer components, different components, or differently arranged components than those shown in FIG. 3. Additionally, or alternatively, a set of components (e.g., one or more components) of device 300 may perform one or more functions described as being performed by another set of components of device 300.

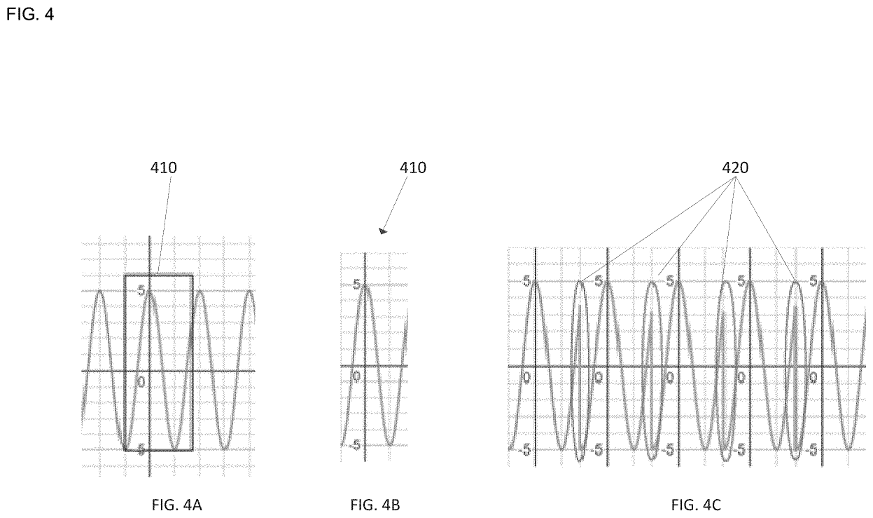

In the real world, a signal such as ECG may include two parts: a real signal and noise. Generally, in the application of noise filtering it is desirable to remove the noise component need as much as possible while minimizing alterations caused by the filter to the real signal. Fast Fourier Transform (FFT) is a popular digital filter schema. FFT assumes the input signal is periodic and continuous, and uses the sum of a series of sine waves to represent the input signal. However, a signal in the real world is usually not periodic and continuous. The input signal of an FFT filter needs to be windowed and appended to be considered as periodic and continuous. For example, such a window 410 is illustrated in FIG. 4. The windowing process usually causes a problem called spectral leakage, which induces unwanted frequency components into the filtered signal and thus alters the information carried by the signal. For example, artifacts such as artifacts 420 may be introduced. Depending on the actual application, this kind of alteration may not be acceptable.

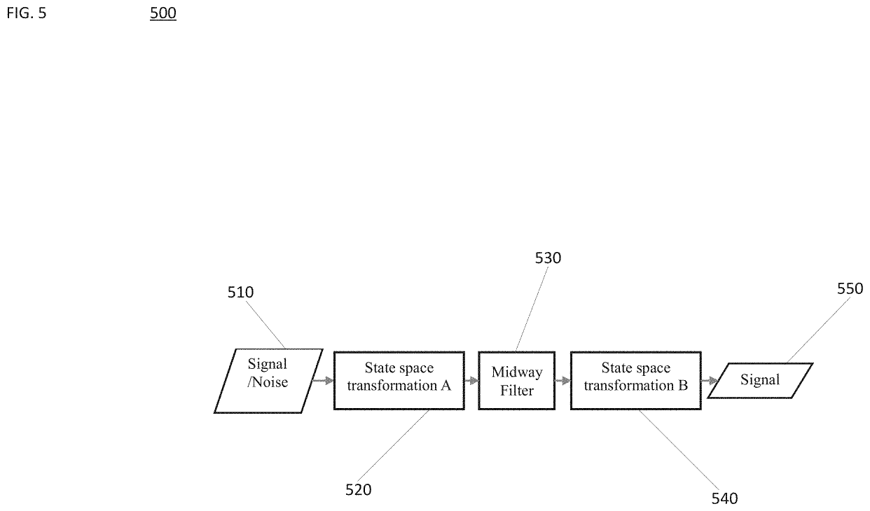

FIG. 5 illustrates a signal filtering schema 500 according to an embodiment. Schema 500 provides a filtering structure that can be used in different scenarios that requires removing or extracting certain component from an input signal 510 which contains noise. Schema 500 may include three steps, as shown in FIG. 5. In step 520, signal 510 is mapped from its original state space into a new state space. In the step 530, a filtering approach is applied in the new state space, and in step 540 the output of the filter is mapped back into the original state space to obtain the filtered signal 550. Schema 500 may remove noise from the input signal as much as possible and while minimizing alterations caused to the real signal. In an embodiment, schema 500 may not require the input signal to be periodic and continuous, and may remove noise/artifacts from an input signal while minimizing the alteration to the real signal. Therefore, the spectral leakage problem of FFT may be avoided. Schema 500 may be used in applications which relate to removal of noise from an input signal without causing significant alteration to the real signal.

According to an embodiment, a filtering schema such as schema 500 may relate to noise filtering in a transformed state space, and the transformation does not assume that the input data is periodic and continuous. Such a filtering schema can be described as follows:

In a first step, which may correspond to step 520, a raw signal S1 may be mapped into a state space as S2 using mathematical transformation A:

S2=A(S1) (Equation 1)

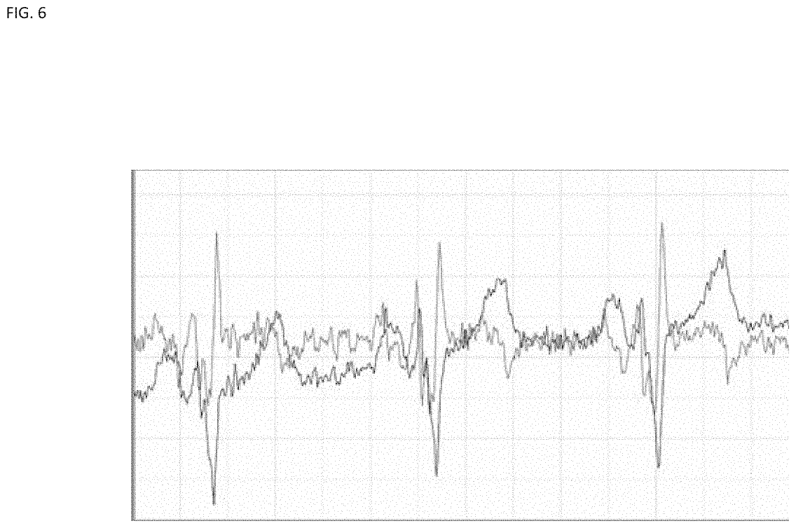

Any transformation that does not require S1 to be periodic and continuous can be used. As an example, the first derivative is used as A, as shown in FIG. 6.

In a second step, which may correspond to step 530, a filtering approach which may be referred to as a midway filter (MF), is applied to S2 to get signal S3:

S3=MF(S2) (Equation 2)

Any filtering approach that does not require periodic and continuous input signal can be used. As an example, the moving average window (MAW) is used as MF.

In the third step, which may correspond to step 540, a mathematical transformation B is applied to S3 to get output signal S4, which is the filtered signal:

S4=B(S3) (Equation 3)

According to an embodiment, B may be an inversed transformation of A. FIG. 4 illustrates a result of the mathematical transformation B, which may be the filtered signal, along with the raw signal.

Because the filtering schema described herein does not assume the input data to be periodic and continuous, the spectral leakage problem may be avoided, and alteration to the real signal may be minimized.

According to an embodiment, the filtering schema can be configured for different application scenarios by choosing different transformations A/B and filtering methods MF as described, and any type of transformation used as A/B and/or any type of filtering method being used as MF may be used in the schema.

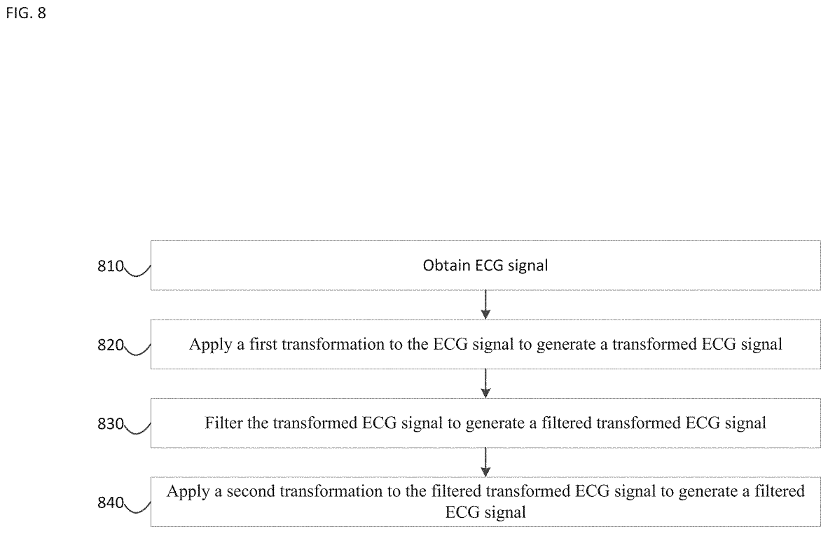

FIG. 8 is a flow chart of an example process 800 for filtering an ECG signal. In an embodiment, process 800 may correspond to the filtering schema discussed above, for example schema 500. In some implementations, one or more process blocks of FIG. 8 may be performed by platform 220. In some implementations, one or more process blocks of FIG. 8 may be performed by another device or a group of devices separate from or including platform 220, such as user device 210.

As shown in FIG. 8, process 800 may include obtaining the ECG signal (block 810).

As further shown in FIG. 8, process 800 may include applying a first transformation to the ECG signal to generate a transformed ECG signal (block 820).

As further shown in FIG. 8, process 800 may include filtering the transformed ECG signal to generate a filtered transformed ECG signal (block 830).

As further shown in FIG. 4, process 800 may include applying a second transformation to the filtered transformed ECG signal to generate a filtered ECG signal (block 840).

In an embodiment, obtaining of the ECG signal may include measuring electromagnetic signals at a surface of a body.

The ECG signal may include at least one from among a P-wave, a PR interval, a PR segment, a QRS complex, an ST segment, a QT interval, and a T-wave.

In an embodiment, the second transformation may include an inverse transformation of the first transformation.

In an embodiment, the ECG signal may be non-periodic.

In an embodiment, the ECG signal may be non-continuous.

In an embodiment, the applying of the first transformation may include obtaining a first derivative of the ECG signal.

In an embodiment, the filtering may include applying a moving average window filter to the transformed ECG signal.

Although implementations herein describe phoneme sequences, it should be understood that other implementations include word sequences, character sequences, and/or the like, as intermediate sequences. In other words, other implementations include the direct mapping between a speech waveform and word and/or character sequences.

Although FIG. 8 shows example blocks of process 800, in some implementations, process 800 may include additional blocks, fewer blocks, different blocks, or differently arranged blocks than those depicted in FIG. 8. Additionally, or alternatively, two or more of the blocks of process 800 may be performed in parallel.

The foregoing disclosure provides illustration and description, but is not intended to be exhaustive or to limit the implementations to the precise form disclosed. Modifications and variations are possible in light of the above disclosure or may be acquired from practice of the implementations.

As used herein, the term component is intended to be broadly construed as hardware, firmware, or a combination of hardware and software.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A method of filtering an electrocardiogram (ECG) signal, the method comprising:
obtaining the ECG signal;
applying a first transformation to the ECG signal to generate a transformed ECG signal;
filtering the transformed ECG signal to generate a filtered transformed ECG signal; and
applying a second transformation to the filtered transformed ECG signal to generate a filtered ECG signal.

2. The method of claim 1, wherein the obtaining of the ECG signal comprises measuring electromagnetic signals at a surface of a body.

3. The method of claim 1, wherein the ECG signal includes at least one from among a P-wave, a PR interval, a PR segment, a QRS complex, an ST segment, a QT interval, and a T-wave.

4. The method of claim 1, wherein the second transformation comprises an inverse transformation of the first transformation.

5. The method of claim 1, wherein the ECG signal is non-periodic.

6. The method of claim 1, wherein the ECG signal is non-continuous.

7. The method of claim 1, wherein the applying of the first transformation comprises obtaining a first derivative of the ECG signal.

8. The method of claim 1, wherein the filtering comprises applying a moving average window filter to the transformed ECG signal.

9. A device for filtering an electrocardiogram (ECG) signal, the device comprising:
at least one memory configured to store program code; and
at least one processor configured to read the program code and operate as instructed by the program code, the program code including:
obtaining code configured to cause the at least one processor to obtain the ECG signal;
first transformation code configured to cause the at least one processor to apply a first transformation to the ECG signal to generate a transformed ECG signal;
filtering code configured to cause the at least one processor to filter the transformed ECG signal to generate a filtered transformed ECG signal;
second transformation code configured to cause the at least one processor to apply a second transformation to the filtered transformed ECG signal to generate a filtered ECG signal.

10. The device of claim 9, further comprising an electromagnetic sensor configured to obtain the ECG signal by measuring electromagnetic signals at a surface of a body.

11. The device of claim 9, wherein the ECG signal includes at least one from among a P-wave, a PR interval, a PR segment, a QRS complex, an ST segment, a QT interval, and a T-wave.

12. The device of claim 9, wherein the second transformation comprises an inverse transformation of the first transformation.

13. The device of claim 9, wherein the ECG signal is non-periodic.

14. The device of claim 9, wherein the ECG signal is non-continuous.

15. The device of claim 9, wherein the first transformation code comprises derivative code configured to cause the at least one processor to obtain a first derivative of the ECG signal.

16. The device of claim 9, wherein the filtering code comprises applying code configured to cause the at least one processor to apply a moving average window filter to the transformed ECG signal.

17. A non-transitory computer-readable medium storing instructions, the instructions comprising: one or more instructions that, when executed by one or more processors of a device for filtering an electrocardiogram (ECG) signal, cause the one or more processors to:
obtain the ECG signal;
apply a first transformation to the ECG signal to generate a transformed ECG signal;
filter the transformed ECG signal to generate a filtered transformed ECG signal; and
apply a second transformation to the filtered transformed ECG signal to generate a filtered ECG signal.

18. The non-transitory computer-readable medium of claim 17, wherein the ECG signal includes at least one from among a P-wave, a PR interval, a PR segment, a QRS complex, an ST segment, a QT interval, and a T-wave.

19. The non-transitory computer-readable medium of claim 17, wherein the ECG signal is non-periodic.

20. The non-transitory computer-readable medium of claim 17, wherein the ECG signal is non-continuous.
